(19) United States
(12) Patent Application Publication
(10) Pub. No.: US 2001/0036463 A1
(43) Pub. Date: Nov. 1, 2001

(54) Transcutaneous immunization for large particulate antigens

(76) Inventors: Richard W. Compans, Atlanta, GA (US); Zhiyi Sha, Brooklyn, NY (US)

Correspondence Address: GREENLEE WINNER AND SULLIVAN P C, 5370 MANHATTAN CIRCLE, SUITE 201, BOULDER, CO, 80303, US

(21) Appl. No.: 09/803,649

(22) Filed: Mar. 9, 2001

Related U.S. Application Data
(60) Provisional application No. 60/188,112, filed on Mar. 9, 2000.

(51) Int. Cl. A61K 39/12; A61K 31/715

(52) U.S. Cl. 424/204.1; 514/54

(57) ABSTRACT

The present Specification discloses noninvasive, economical and easy methods for inducing an immune response against a particulate antigen; i.e., by transcutaneous administration of the particulate antigen such as a virus particle which has desirably been inactivated or attenuated so as not be result in a disease in the person or animal to which it has been administered. Advantageously, the immunogenic composition comprises a sialic binding component (such as a viral hemagglutinin) included as part of or in addition to the particulate antigen. One such example of a particulate virus is influenza virus, desirable having been formalin-inactivated.

### CROSS REFERENCE TO RELATED APPLICATIONS

This application claims priority from U.S. Provisional Application No. 60/188,112, filed Mar. 9, 2000.

### ACKNOWLEDGMENT OF FEDERAL RESEARCH SUPPORT

This invention was made, at least in part, with funding from the National Institutes of Health (Grant No. AI 43045 and Grant No. AI 43068). Accordingly, the United States Government has certain rights in this invention.

### BACKGROUND OF THE INVENTION

The field of the invention is the area of immunology, especially as related to compositions for eliciting an immune response to a particulate antigen without the need for puncturing or breaking the skin, such as with a viral particle, as specifically exemplified by an influenza virus.

Transcutaneous immunization is a relatively new approach for vaccine delivery. In this approach, antigens are topically applied to the intact skin, without the help of needles, as used in conventional immunization techniques. It has been recently reported that applying solutions containing cholera toxin together with tetanus or diphtheria toxoids results in potent antibody responses against the cholera toxin and the co-administered antigens (Glenn et al. (1998) Nature 391:851; Glenn et al. (1998) J. Immunol. 161:3211-3214; Glenn et al. (1999) Infect. Immun. 67:1100-1106). Similarly, U.S. Pat. No. 5,980,898 Glenn et al., 1999) teaches the use of an adjuvant, such as cholera toxin, in compositions for transcutaneous inmunization. All the examples in that patent utilized antigens which were soluble proteins.

There is a longfelt need in the art for technically simple, effective and economical methods for delivering immunogenic compositions to human and animal patients, especially compositions for administering viral antigens to large numbers of people.

### SUMMARY OF THE INVENTION

The present invention provides noninvasive methods for inducing an immune response in a human or animal. Specifically, a particulate antigen is administered onto the unbroken skin of a human or animal in whom the immune response is desired. Such transcutaneous administration of a particulate antigen results in at least a humoral response specific to at least one component of the particulate antigen. The particulate antigen can be a virus particle, a virus-like particle, a mycoplasma cell, a bacterial cell, membranous preparation, and desirably where the particle is a virus or a cell, the preparation has been treated to inactivate any ability to replicate or to result in an otherwise harmful effect on the human or animal. Virus particles can include, but are not limited to, orthomyxoviruses and paramyxoviruses and others, including influenza virus, parainfluenza virus, a hepatitis virus, measles virus, vaccinia virus, herpes virus, rhinovirus. Desirably the virus particle has a sialic acid binding moiety on its surface. Viruses which have a sialic acid binding surface component include the orthomyxoviruses and the parainfluenza viruses. Influenza virus is an important specific example. In addition, mixed virus particles can be engineered to display a sialic acid binding component on their surfaces, for example a hemagglutinin derived in terms of coding sequence from influenza virus. In the case of such engineered virus particles, the particles can also contain antigenic determinants of viruses including other enveloped viruses (including noninfectious HIV, SIV, FIV and others) and those viruses with glycoproteins having terminal sialic acid residues. Additional virus examples are vesicular stomatitis virus, rabies virus, measles virus, flavivirus, and alphaviruses and herpes viruses.

### BRIEF DESCRIPTION OF THE DRAWINGS

FIG. 1 shows the antibody responses in sera of mice transcutaneously immunized with inactivated influenza virus PR8 as described hereinbelow. The sera were collected 4 weeks after the second immunization. IgG1 antibody concentration (expressed as μg/ml) was determined using standard ELISA assays and commercially available antibody specific for mouse IgG. Control mice were mock-immunized with PBS, and PR8 mice were immunized as described.

FIG. 2 presents the results of plaque neutralization assays carried out with sera from mice transcutaneously immunized with inactivated influenza virus PR8. Sera were collected 4 weeks after the second immunization. (●) Control mice were mock-immunized with PBS, (♦) PR8 mice were immunized with 50 μg of influenza virus PR8 as described, (▪) mice immunized by intranasal administration of 10 μg influenza virus PR8, and (▴) mice challenged with a sublethal dose of intact influenza virus PR8.

FIG. 3 shows protection of mice immunized transcutaneously with inactivated influenza virus PR8 against a lethal dose challenge of live influenza virus PR8. Transcutaneously immunized mice were challenged intranasally with 10×LD50 of live PR8 virus 4 weeks after the second immunization. (▪) mice which were mock-immunized with PBS, and (●) mice which received transcutaneous administration of the inactivated virus.

### DETAILED DESCRIPTION OF THE INVENTION

The skin of humans and animals is generally composed of an inner layer (the dermis)which is vascular in nature and which contains neural networks, secretory glands (e.g., oil glands) and excretory glands (e.g., sweat glands) and in certain cases, hair and hair follicles and nails, and an outer layer (the epidermis). The outer layer is thinner, and it primarily contains stratified keratinized cells. The outermost portion of the epidermis is 15-20 cells thick, and the intercellular spaces are filled with lipids, especially cholesterol, ceramide and fatty acids.

Transcutaneous immunization has been demonstrated with protein antigens, i.e. with cholera toxin as antigen or for protein antigens administered together with cholera toxin as an immunological adjuvant. A transcutaneous immunization system delivers antigens to specialized cells, e.g., antigen presenting cells, dendritic cells or lymphocytes, that produce an immune response.

Whereas the cholera toxin has a molecular weight of about 87 kDa, an influenza virus particle has a particle weight of about 250,000 kDa and a particle diameter of about 100 nm. This virus had a weight nearly 3000-fold larger than that of cholera toxin or bovine serum albumin. Surprisingly, the virus particles could penetrate the unbroken skin and come in contact with immune cells so that not only virus-specific immunoglobulins were produced, but protective immunity also resulted, in the absence of any adjuvant.

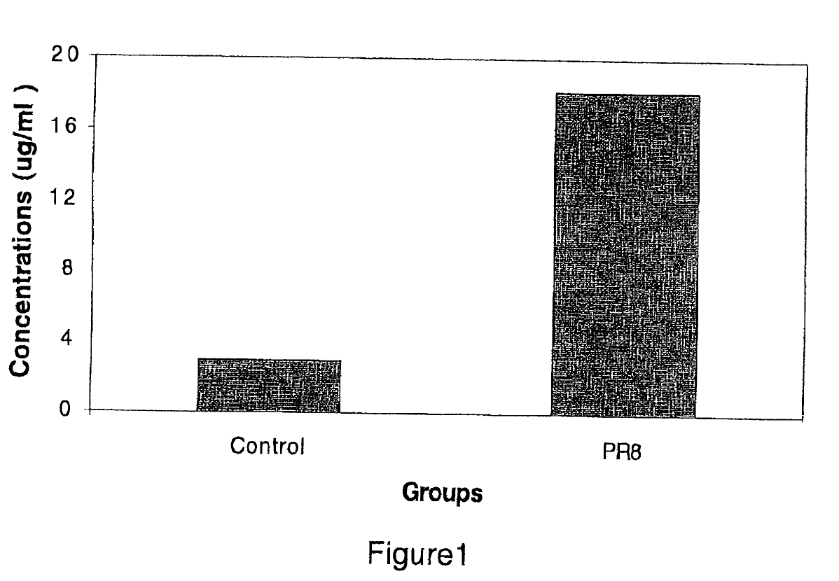

To investigate the possibility that intact virus particles could induce an immune response, the present inventors prepared formalin-inactivated influenza PR8 virus particles. The intact formalin-inactivated virus particles were applied to the shaved skin of C57BL/6 mice, in a formulation which did not comprise cholera toxin as an adjuvant. The magnitude of virus-specific antibody responses was evaluated by measuring PR8-specific immunoglobulin concentrations in sera using an ELISA assay as described in Pertmer et al. (1996) J. Virol. 6119-6125. The results are shown in FIGS. 1-3. Surprisingly, not only was there significant production of immunoglobulins specific for influenza virus protein, but there was also protective immunity to challenge (nasal route) with the cognate live, virulent influenza virus.

The present invention provides noninvasive methods for inducing an immune response in a human or animal. Specifically, a particulate antigen is administered onto the unbroken skin of a human or animal in whom the immune response is desired. Such transcutaneous administration of a particulate antigen results in at least a humoral response specific to at least one component of the particulate antigen. The particulate antigen can be a virus particle, a virus-like particle, a mixed virus-like particle, a mycoplasma cell, a bacterial cell, a fragment of a bacterial cell, membraneous preparation, and desirably where the particle is a virus or a cell, the preparation has been treated to inactivate any ability to replicate or to result in an otherwise harmful effect on the human or animal. Virus particles can include, but are not limited to, influenza virus, parainfluenza virus, a hepatitis virus, measles virus, vaccinia virus, herpes virus, rhinovirus. Desirably the virus particle has on its surface a sialic acid binding component. Viruses which naturally include a sialic acid binding component include the paramyxoviruses and the orthomyxoviruses. A clinically important example is influenza virus, in which the sialic acid binding component is the hemagglutinin. In addition virus particles can be engineered to contain a sialic acid binding component, for example, the hemagglutinin derived from an influenza virus. Mixed virus particles or virus-like particles can be made using well known molecular biological technology by engineering the cells in which the virus particles or virus-like particles are made to also express a sialic acid binding protein, for example the hemagglutinin of influenza virus, with the result that the sialic binding component is incorporated on the surface of the particle.

As an alternative to mixing a sialic acid containing antigen with a sialic acid binding paramyxovirus, e.g., influenza virus, preparation, one of ordinary skill in the art can prepare phenotypically mixed virus-like particles, which particles comprise the sialic acid binding component (e.g., hemagglutinin) as well as the desired antigen. See, for example, Vzorov and Compans (2000) J. Virol. 74:8219-8225, for a discussion of mixed virus-like, non-infectious particles and incorporation of human influenza virus hemagglutinin and optionally the fusion (F) protein or the corresponding proteins from human parainfluenza virus. Cloning and expression of parainfluenza virus hemagglutinin and F protein are described, inter alia, in Sequences encoding hemagglutinins from influenza viruses and parainfluenza viruses are known to the art (e.g., on GenBank). Such noninfectious particles can be used in immunogenic compositions for transcutaneous administration. Phenotypically mixed viruses can also be prepared by coexpression of paramyxovirus coding sequences including the sialic acid binding component and the sialic acid containing antigens of another virus (e.g., those genes required for the production of virus like particles). For discussion of production of virus like particles of retroviruses, see U.S. Pat. No. 6,077,662 and references cited therein.

An antigen can comprise carbohydrate, glycoprotein, lipid, lipoprotein, phospholipid, protein, nucleic acid, conjugates of one or more of the foregoing molecules or any other material known to induce an immune response. In the present context, the antigen is in the form of particulate material which, in turn, is composed of one or more of the aforementioned molecules. As specifically exemplified herein, the particulate antigen is a virus particle, desirably the virus particles are attenuated in virulence or inactivated by heat, ultraviolet irradiation or chemical treatment such as formalin or psoralen treatment, so that infection does not result from the entry of the virus particle or cell into or through the skin.

Immunogenic particles can be applied to the skin in a composition containing about 10 to about 250 μg/ml, about 25 to 200, or about 50 to about 100 μg/ml of particle protein. A volume from about 40 μl to about 500 μl is applied, desirably about 50 to about 500 μl/ml is applied to about 1 to about 8 cm². Desirably the particles are suspended in a pharmaceutically acceptable buffer with a pH from about 5.5 to about 8.0, desirably from about 8.0. Phosphate buffered saline (pH about 7.4) is a preferred buffer. The ionic strength is advantageously similar to that of intracellular fluid.

Optionally, an occlusive patch can be applied over the skin over the site where the particle-containing immunogenic composition has been applied. The patch can serve the function of keeping the area moist, or the patch can release skin penetration enhancing compounds. Penetration enhancers are described in U.S. Pat. Nos. 4,948,588 and 5,785,978, for example.

Processes for preparing a formulation for use in the methods of the present invention are well-known to the art; the antigen of interest is combined with a pharmaceutically acceptable carrier and optionally, with a skin-penetrating agent and/or an adjuvant, provided that the adjuvant is not cholera toxin or a cholera toxoid protein. Suitable carriers are described in, e.g., Remington's Pharmaceutical Sciences, E. W. Martin. Such formulations contain an amount of the antigen effective for inducing an immunological response and a suitable proportion of the carrier. The formulation can be applied to the skin of the human or animal in the form of a cream, emulsion, lotion, gel, ointment, paste, suspension or any other form known to the art which allows migration of the particulate antigen through the unbroken skin. Advantageously, the formulation can further comprise components which promoter hydration of the skin, and/or penetration of the skin. Other optional additives can include diluents, binders, stabilizers, preservatives to maintain microbial quality and coloring agents.

Increasing the hydration of the stratum corneum tends to increase the rate of percutaneous adsorption of a given compound (Robin and Walker, 1993). As used in the present Specification, penetration enhancers include water, physiological buffers, saline solutions or alcohols which do not perforate the skin.

Without wishing to be bound by theory, it is believed that transcutaneous administration of an immunogenic composition or formulation carries the particulate antigen to the cells of the immune system in which an immune reaction is produced. The antigen passes through the protective outer layers of the skin and induces the immune response directly or indirectly to an antigen presenting cell such as a macrophage, a tissue macrophage, Langerhans cell, dendritic cell, dermal dendritic cell, B lymphocyte or Kupffer cell. Alternatively, or in addition, the antigen may pass through the stratum corneum via a hair follicle or a skin organelle such as a sweat gland or an oil gland.

Immune responses to transcutaneously administered soluble proteins, especially cholera toxin and ADP-ribosylating toxins (such as diphtheria toxin), have been shown to activate Langerhans cells and to stimulate a humoral response with the production of IgG, IgM, IgA but not IgE.

The present invention provides several distinct and important advantages over traditional methods of administering immunogenic compositions to large numbers of humans or animals. The present invention encompasses the topical application of inactivated virus preparations on the unbroken skin of the individuals. The economics of vaccination are greatly improved because this technique does not rely on sterile needles or other medical implements which breach the skin. Similarly, the lack of a need for sterile implements allows greater speed of administration, with less medical sophistication required, and it can increase the likelihood of effective administration in economically deprived areas and in areas where there is poor access to medical supplies. Other advantages include less emotional distress to the persons or animals receiving the immunogenic compositions, reduced danger of infections associated with contaminated needles or other sharp objects, and simpler disposal of the medical waste associated with the process because of the absence of sharp materials.

As used herein, a recombinant protein is one which is produced in a cell as the result of genetically engineering that cell to produce a protein of interest, where the coding sequence for the protein is operably linked to nucleic acid sequences with which it is not associated in nature.

With respect to “derived from” in the present context, it is intended that this be interpreted to mean that a recombinant protein derived from a particular source is one for which the coding sequence has been excised from the source and later used to create a new nucleic acid molecule which can be used to produce the protein or the nucleotide sequence encoding the protein in the source (organism or virus) has been used to produce a new nucleic acid molecule which can be used to produce the protein.

In the context of the present invention, unbroken skin means the external skin of a human or animal, and this language is not intended to encompass the mucosal surfaces with the nostrils or nose, trachea, bronchi, lungs, mouth, esophagus, stomach, intestines, rectum or urogenital system.

Monoclonal or polyclonal antibodies, preferably monoclonal, specifically reacting with an antigen or particulate material of interest can be made by methods known in the art. See, e.g., Harlow and Lane (1988) Antibodies: A Laboratory Manual, Cold Spring Harbor Laboratories; Goding (1986) Monoclonal Antibodies: Principles and Practice, 2d ed., Academic Press, New York; and Ausubel et al. (1993) Current Protocols in Molecular Biology, Wiley Interscience, New York, N.Y.

Standard techniques for cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described in Sambrook et al. (1989) Molecular Cloning, Second Edition, Cold Spring Harbor Laboratory, Plainview, N.Y.; Maniatis et al. (1982) Molecular Cloning, Cold Spring Harbor Laboratory, Plainview, N.Y.; Wu (ed.) (1993) Meth. Enzymol. 218, Part I; Wu (ed.) (1979) Meth. Enzymol. 68; Wu et al. (eds.) (1983) Meth. Enzymol. 100 and 101; Grossman and Moldave (eds.) Meth. Enzymol. 65; Miller (ed.) (1972) Experiments in Molecular Genetics, Cold Spring Harbor Laboratory, Cold Spring Harbor, N.Y.; Old and Primrose (1981) Principles of Gene Manipulation, University of California Press, Berkeley; Schleif and Wensink (1982) Practical Methods in Molecular Biology; Glover (ed.) (1985) DNA Cloning Vol. I and II, IRL Press, Oxford, UK; Hames and Higgins (eds.) (1985) Nucleic Acid Hybridization, IRL Press, Oxford, UK; Setlow and Hollaender (1979) Genetic Engineering: Principles and Methods, Vols. 1-4, Plenum Press, New York; and Ausubel et al. (1992) Current Protocols in Molecular Biology, Greene/Wiley, New York, N.Y. Abbreviations and nomenclature, where employed, are deemed standard in the field and commonly used in professional journals such as those cited herein.

All references cited in the present application are incorporated by reference herein to the extent that there is no inconsistency with the present disclosure.

The following examples are provided for illustrative purposes, and are not intended to limit the scope of the invention as claimed herein. Any variations in the exemplified articles which occur to the skilled artisan are intended to fall within the scope of the present invention.

### EXAMPLES

### Example 1. Influenza Virus Preparation

Influenza virus stain PR8 virions were purified and inactivated with formalin as described in Novak et al. (1993) Vaccine 11:55-60. After inactivation was complete, the formalin is removed by dialysis. The virus concentration in the suspension used in the immunization experiments is adjusted to range of 50-100 μg protein per ml in a buffer suitable for use on the unbroken skin, for example, phosphate buffered saline, with a pH from about 5.5 to about 8.0, desirably between about 6.0 and about 8.0. PBS is a suitable pharmaceutically acceptable carrier for the antigenic particles of the present immunization methods.

### Example 2. Administration of Particulate Antigens

Formalin-inactivated influenza virus was used as the test antigen in the demonstration of transcutaneous immunization using a particulate antigen. 8-12 week old C57BL/6 mice were shaved on the dorsum over a 2 cm² area. 80 μl of a suspension containing 50 μg intact formalin-inactivated influenza virus PR8 were applied to the shaved areas four times, using 20 μl each time. Those areas were rubbed with the side of the pipet tips for 20 sec each time the immunogenic compositions were administered. The mice were immunized a second time in the same manner 4 weeks after the first administrations. Serum samples were collected 4 weeks after the last administration. IgG1 antibody concentrations (μg/ml) were determined using a standard ELISA and commercially available IgG1-specific antibody. Control mice received mock immunizations of PBS.

### Example 3. Virus Neutralization Assay

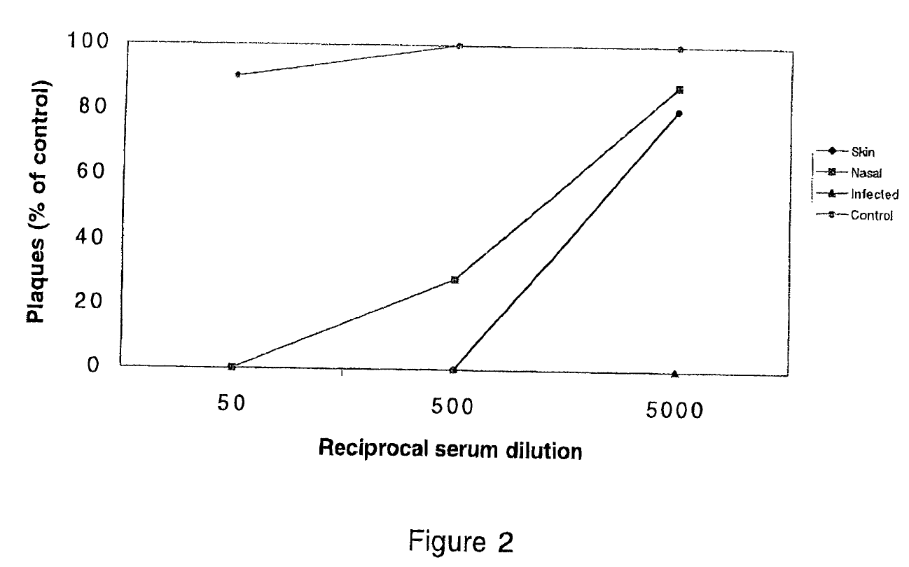

To determine whether transcutaneous administration of the formalin-inactivated influenza virus PR8 results in the production of neutralizing antibodies, serum was collected from mice one month after immunization after the transcutaneous administration of the inactivated virus containing composition. There was a prior boost one month after the initial immunization. Approximately 100 plaque forming units in 200 μl of PR8 virus grown in embryonic egg were incubated with sera at different dilutions, and a standard plaque reduction assay was carried out using MDCK cells. Sera from transcutaneously immunized mice showed virus neutralizing activity at a 1:1000 dilution, which is comparable to the neutralizing activity in mice immunized via the intranasal route with formalin-inactivated influenza virus PR8. In contrast, the sera from unimmunized mice showed no virus neutralizing activity. See FIG. 2.

### Example 4. Protective Immunity

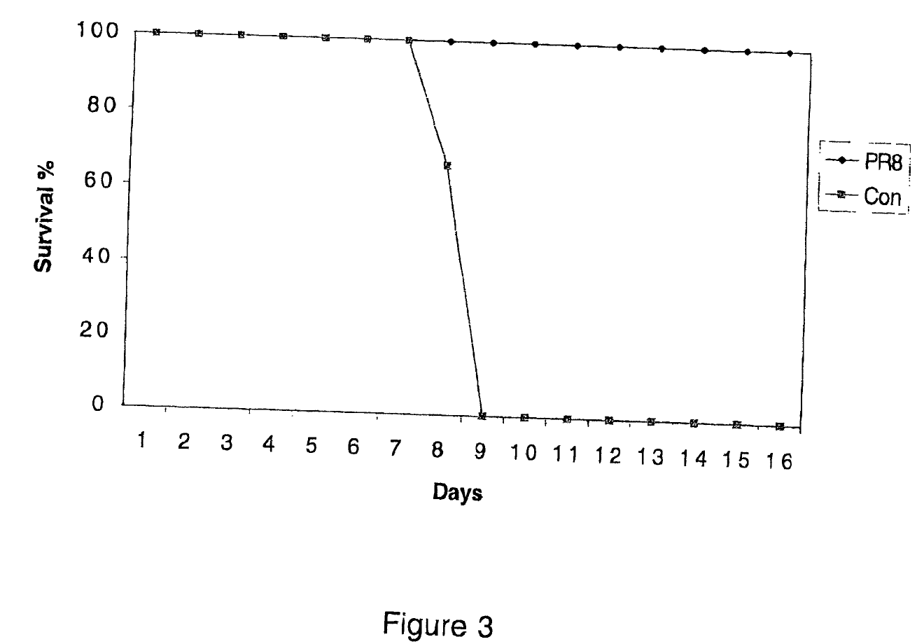

To determine whether the in vitro virus neutralizing activity is indicative of an in vivo protective effect, the transcutaneously immunized mice were challenged with live influenza virus PR8, at a dose 10 times the LD50 for this virus. The challenge virus were administered intranasally under anesthesia. The immunized mice were found to be fully protected from the live virus challenge. All the immunized mice survived, and none of these mice experienced weight loss or decreased physical activity. By contrast, the control (unimmunized) C57BL/6 mice all died at 6-8 days after the virus challenge. See FIG. 3, in which the results obtained from the mouse model indicate that protective immunity results from the transcutaneous administration of influenza virus.

We claim:

1. A method for inducing an immune response comprising the step of applying to the unbroken surface of the skin a composition comprising antigenic particles and a pharmaceutically acceptable carrier, wherein said composition does not also comprise cholera toxin or a cholera toxoid protein.

2. The method of claim 1 wherein the antigenic particles are inactivated virus particles.

3. The method of claim 2 wherein the particles are characterized by a diameter from about 10 to about 250 nm.

4. The method of claim 3 wherein the antigenic particles are from about 50 to about 200 nm in diameter.

5. The method of claim 4 wherein the antigenic particles are about 100 nm in diameter.

6. The method of claim 5 wherein the inactivated virus particles contain a sialic acid binding component.

7. The method of claim 6 wherein the inactivated virus particles are selected from the group consisting of orthomyxovirus particles and paramyxovirus particles.

8. The method of claim 7 wherein the inactivated virus particles are influenza virus particles.

9. The method of claim 1 wherein the antigenic particles are virus-like particles which comprise a sialic acid binding component.

10. The method of claim 9 wherein the sialic acid binding component is a sialic acid specific hemagglutinin.

11. The method of claim 10 wherein the sialic acid binding component is incorporated into the particles by mixed infection with an orthomyxovirus or a paramyxovirus and a virus of interest.

12. The method of claim 1 wherein the virus particles are mixed virus particles comprising a sialic acid binding component which is heterologous to the virus.

13. The method of claim 12 wherein the sialic acid binding component is a recombinant hemagglutinin of influenza virus or parainfluenza virus.

14. The method of claim 12 where the sialic acid binding component is incorporated through mixed infection with an orthomyxovirus or a paramyxovirus and a virus of interest.

15. The method of claim 12 wherein the virus particles are noninfectious particles of parainfluenza virus, hepatitis C virus, hepatitis virus B, measles virus, vaccinia virus, herpes virus or respiratory syncytium virus.

16. The method of claim 2 wherein the virus particles have been inactivated by chemical treatment, ultraviolet irradiation, heat treatment or psoralen treatment.

17. The method of claim 16 wherein the chemical treatment is formalin treatment.

18. A method for inducing an immune response comprising the step of applying to the unbroken surface of the skin a composition comprising live virus particles and a pharmaceutically acceptable carrier, wherein said composition does not also comprise cholera toxin.

19. The method of claim 18 wherein the live virus particles are attenuated virus particles.

20. The method of claim 18 wherein said virus particles comprise a sialic acid binding component.

21. The method of claim 20 wherein the sialic acid binding component is a hemagglutinin.

22. The method of claim 21 wherein the hemagglutinin is derived from an orthomyxovirus or a paramyxovirus.

23. The method of claim 22 wherein the hemagglutinin is derived from influenza virus or a parainfluenza virus.
